(19) United States
(12) Patent Application Publication
(10) Pub. No.: US 2021/0299335 A1
(43) Pub. Date: Sep. 30, 2021

(54) METHOD FOR DYNAMIC FILTRATION OF A CROSS-LINKED HYDROGEL

(71) Applicant: MERZ PHARMA GMBH & CO. KGAA, Frankfurt am Main (DE)

(72) Inventors: Michael PFEIL, Koenigstein (DE); Wolfgang KESSLER, Berlin (DE); Manuel CONRAD, Dessau Rosslau (DE); Bjoern NIEMCZAK, Leipzig (DE); Patrik VUKOVIC, Berlin (DE)

(21) Appl. No.: 17/263,973

(22) PCT Filed: Aug. 6, 2019
(86) PCT No.: PCT/EP2019/071096

§ 371 Date: Jan. 28, 2021

(30) Foreign Application Priority Data
Aug. 7, 2018 (EP) PCT/EP2018/071328

(51) Int. Cl. A61L 27/52; A61L 27/20; A61L 27/54; A61K 31/167; C08B 37/08; B01D 69/06; B01D 61/14; B01D 69/02; B01D 63/16

(52) U.S. Cl. CPC A61L 27/52; B01D 2315/02; A61L 27/54; A61K 31/167; C08B 37/0072; B01D 69/06; B01D 61/145; B01D 69/02; B01D 63/16; A61L 2300/402; A61L 2430/34; A61L 2300/204; B01D 2313/243; B01D 2315/16; B01D 2325/02; A61L 27/20

(57) ABSTRACT

The present invention relates to a method for dynamic filtration of a cross-linked biopolymer-based hydrogel to remove unwanted molecules from the gel. In particular, the invention relates to dynamic filtration of a hyaluronic acid hydrogel using a dynamic filtration construction with rotating and semipermeable filter discs.

### FIELD OF THE INVENTION

The present invention relates to a method for dynamic filtration of a cross-linked biopolymer-based hydrogel to remove unwanted molecules from the gel. In particular, the invention relates to dynamic filtration of a hyaluronic acid hydrogel using a dynamic filtration construction with rotating and semipermeable filter discs.

### BACKGROUND OF THE INVENTION

Hyaluronic acid is a polymer natural to the body, which since some time is employed in medicine in different fields such as in orthopedics and in ophthalmology. Nowadays, hyaluronic acid is increasingly used in aesthetic medicine and in plastic surgery. The broad application of hyaluronic acid is particularly due to its very high binding capability of water. In aqueous medium, even at low concentration of hyaluronic acid, viscoelastic gels are formed, which are biologically degradable, and which have advantageous properties.

Pure hyaluronic acid is relatively fastly degraded in the human body. For this reason, hyaluronic acid molecules are often chemically cross-linked with one another, whereby the degradation is severely decreased, and the desired effects are maintained for a period of about six to twelve months. Thus, cross-linked hyaluronic acid gels are, among others, frequently used in the aesthetic medicine for the treatment of wrinkles, contouring and volume increase of the upper and lower lip, the improvement of the contours of the cheeks and chin, and for corrections of the nose, etc.

The known hyaluronic acid gels can be roughly classified in two different types, namely mono-phased and bi-phased gels. The mono-phased hyaluronic acid gels consist of a single phase and are non-particulate. The preparation of such mono-phased gels is disclosed, for example, in WO 2008/068297, U.S. Pat. Nos. 8,450,475, 8,455,465, 7,741,476, and 8,052,990. Known commercial mono-phased hyaluronic acid gels are, for example, Juvéderm®, Teosyal®, Glytone Professional®, and the mono-phased, double-cross-linked hyaluronic acid gels, which are known as Belotero®, Esthélis®, Fortélis®Extra and Modélis®Shape.

The bi-phased hyaluronic acid gels comprise a cross-linked hyaluronic acid material, which is dispersed in a liquid phase. Such gels are particulate and may be made as described in, for example, EP 0 466 300. Commercially available bi-phased gels are, for example, Hylaform®, Restylane®, and Perlane®.

The making of cross-linked hyaluronic acid gels occurs by means of a multi-step process and is described in more detail in WO 2005/085329.

Methods of making gels based on polysaccharides such as hyaluronic acid are further disclosed in WO 2014/064633, US 2013/210760, US 2013/203696, WO 2010/115081, WO 2012/062775, WO 2013/185934, WO 2009/077399 and WO 2008/034176.

The conventional methods for dialysis of hyaluronic acid gels frequently require a relatively high manual time and effort. This is insofar disadvantageous as quality and reproducibility are decreased and production time and costs are increased. Furthermore the risk of microbial contamination increases with process time of the dialysis step.

Frequently, 1,4-butanediol diglycidyl ether (BDDE) is used as a crosslinking agent for hyaluronic acid hydrogels. However, BDDE may be toxic and there is a demand to produce hydrogels having less unwanted molecules.

### OBJECT OF THE INVENTION

The present invention is based on the object to provide an improved method to remove unwanted molecules which are generated during production of a cross-linked biopolymer-based hydrogel.

### SUMMARY OF THE INVENTION

The invention relates to a method for dynamic filtration of a cross-linked biopolymer-based hydrogel using a dynamic filtration device which is equipped with semipermeable filter disc(s) to remove unwanted molecules comprising the steps of i) concentrating the gel by applying a rotational speed within the range of 20 1/min to 500 1/min and a overpressure within the range of 0.5 to 6 bar to a predetermined concentration; or pumping the gel directly into the process chamber of the dynamic filtration device; ii) conducting a diafiltration to reduce unwanted molecules by applying a rotational speed within the range of 20 1/min to 500 1/min and a overpressure within the range of 0.5 to 6 bar.

### FIGURES

FIG. 1: Frequency sweep comparison between a gel which is processed according to the invention (“DCF gel”; storage modulus (G′) 200 Pa) and a gel which is processed according to a standard method (“standard gel”; storage modulus (G′) 216 Pa).

1 (dotted line)=DCF gel: G′
2 (continuous line): standard gel: G′
3 (dotted line)=DCF gel: G″
4 (continuous line): standard gel: G″
5 (dotted line)=DCF gel: complex viscosity
6 (continuous line)=standard gel: complex viscosity

FIG. 2: Amplitude sweep comparison between a gel which is processed according to the invention (“DCF gel”; storage modulus (G′) 241 Pa) and a gel which is processed according to a standard method (“standard gel”; storage modulus (G′) 249 Pa). Cross-over point=flow-point

1 (dotted line)=DCF gel: G′
2 (continuous line): standard gel: G′
3 (dotted line)=DCF gel: G″
4 (continuous line): standard gel: G″

FIG. 3:

### 1: DCF gel: G′

### 2: DCF gel G″

3: DCF gel: complex viscosity

FIG. 4: Frequency sweep of the gel according to example 3/trial 4

continuous line with triangle ▴: loss modulus G″
continuous line with square ▪: storage modulus G′
continuous line with circle ●: complex viscosity [q*]

FIG. 5: Amplitude sweep of the gel according to example 3/trial 4

continuous line with triangle ▴: loss modulus G″
continuous line with square ▪: storage modulus G′
cross over point G″=G′: shear stress 440 Pa; storage modulus 54 Pa

FIG. 6: Frequency sweep of the gel according to example 3/trial 5

continuous line with triangle ▴: loss modulus G″
continuous line with square ▪: storage modulus G′
continuous line with circle ●: complex viscosity [q*]

FIG. 7: Amplitude sweep of the gel according to example 3/trial 5

continuous line with triangle ▴: loss modulus G″
continuous line with square ▪: storage modulus G′
cross over point G″=G′: shear stress 350 Pa; storage modulus 98 Pa

### DETAILED DESCRIPTION OF THE INVENTION

The present invention may be understood more readily by reference to the following detailed description of the invention and the example included therein.

The term “dynamic filtration device” as used herein refers to a means using a hollow filter element in the form of a plate or disk which is arranged movable on a hollow shaft. By the relative movement of the element turbulence arise in the medium to be filtered, whereby the surface of the filter element is cleaned. Such a filter device is described for example in WO 00/47312. The rotating filter element allow for decoupling of overflow velocity from transmembrane pressure (TMP). The combination of centrifugal and shear forces in dynamic filtraton modules improves control of cover layer build-up. By using this technology the formation of a clogging layer is reduced. In addition, the relative movement ensures a mixing of the retentate in the filter machine. Such a dynamic filtration device can be obtained from different companies, for example from Andritz AG (Headquarters Graz Austria). The key element of the dynamic filtration device is the membrane disk. Its diameter, number of discs and alignment may vary depending on the intended scale.

According to the invention, the method of dynamic filtration can be applied on various cross-linked biopolymer-based hydrogels (also referred to as “hydrogel(s)” in the following). Methods for the production of cross-linked biopolymer-based hydrogels are well known in the art, for example, the making of cross-linked hyaluronic acid gels is described in WO 2005/085329 A1. Further methods of making gels based on polysaccharides are disclosed in WO 2014/064633, US 2013/210760, US 2013/203696, WO 2010/115081, WO 2012/062775, WO 2013/185934, WO 2009/077399 and WO 2008/034176.

In one aspect, the invention relates to a method for dynamic filtration of a cross-linked biopolymer-based hydrogel comprising the steps of

a) transferring a cross-linked biopolymer-based hydrogel in a dynamic filtration device which is equipped with semipermeable filter disc(s) and diafiltrating the gel comprising the steps of:
i) concentrating the gel by applying a rotational speed within the range of 20 1/min to 500 1/min and a overpressure within the range of 0.5 to 6 bar to a predetermined concentration; or pumping the gel directly into the process chamber of the dynamic filtration device;
ii) conducting a diafiltration to reduce unwanted molecules by applying a rotational speed within the range of 20 1/min to 500 1/min and a overpressure within the range of 0.5 to 6 bar;
   b) optionally adding a mixture comprising a non-cross-linked polymer, in particular non cross-linked hyaluronic acid, and water to the gel.

As used herein, the term “diafiltration” is used to mean a continuous diafiltration (also referred to as constant volume diafiltration) which involves washing out the original buffer salts or other low molecular weight species in the retentate (sample) by adding water or a new buffer to the retentate at the same rate as filtrate is being generated.

The term “unwanted molecules” as used herein refers to unreactive or unbound polymer cross-linker molecules (e.g. BDDE) and their degradation products.

Surprisingly, it has been found that the method according to the invention removes significantly unwanted molecules from the gel, while the structural stability of the gel was maintained in spite of the share stress arising from the rotating filter discs. In addition, it was surprisingly found that the required process time and required buffer for conducting the method according to the invention was significantly reduced in comparison to conventional dialyses. It is a further advantage of the method according to the invention that flux rates are increased (two to ten times) in comparison to conventional cross-flow filtrations and that the retentate can be higher concentrated. These advantages lead to an improved method to remove unwanted molecules from cross-linked and highly viscous hydrogels.

The biopolymer-based hydrogel is made of a biologically degradable polymer, for example: hyaluronic acid, heparosan, alginate, pectin, gellan gum, chondroitin sulfate, keratan, keratan sulfate, heparin, heparin sulfate, cellulose, chitosan, carrageenan, xanthan, or salts or derivatives thereof or combinations thereof. The term “hyaluronic acid” is synonymously used with the term “hyaluronan”.

Although in the following the present invention is described by means of a hyaluronic acid hydrogel, this does not mean that the disclosure is in any way limited to a hyaluronic acid hydrogel. Rather, any biopolymer-based hydrogel may be used in place of hyaluronic acid hydrogel, in particular hydrogels which are made of one of the above-mentioned exemplary polymers.

The biopolymer-based hydrogel is cross-linked by means of a chemical cross-linking agent and the hydrogel contains a surplus of the used chemical cross-linking agent. The surplus of the chemical cross-linking agent is removed from the hydrogel by the method according to the invention. In addition, other unwanted molecules may also be removed from the hydrogel by the method according to the invention.

The term “cross-linker” comprises all compounds bearing at least two groups, which are capable of reacting with one or more of the functional groups of the biopolymer-based hydrogel and to cross-link same, for example with the hydroxyl groups. Suitable groups of the cross-linker for cross-linking may be selected from carboxyl, epoxide, halogen, vinyl, isocyanate, or protected isocyanate groups. Exemplary compounds, which may be used as cross-linker, are, for example, 1,4-butanediol diglycidyl ether (BDDE), polyvinylsilane (PVS), PEG-based crosslinking agents, divinyl sulfone (DVS). In particular embodiments, the method according to the invention removes unbound or surplus of 1,4-butanediol diglycidyl ether (BDDE) and/or other unwanted molecules from the hydrogel.

Preferably, the cross-linked biopolymer-based hydrogel (in particular the hydrogel made of hyaluronic acid) exhibits an initial concentration in the range of from 10 to 70 mg/g, preferably from 20 to 50 mg/g. In terms of the present invention the concentration of the hydrogel means the amount of the cross-linked biopolymer (given in mg) based on the total amount of the hydrogel (given in g). Typically, the initial concentration is the concentration of the hydrogel which is transferred in the dynamic filtration device in step a) and which is optionally concentrated or pumped directly into the process chamber in step i).

In another embodiment of the invention the hydrogel can be diluted (e.g. using a buffer solution applied in the diafiltration) before it is transferred into the dynamic filtration device, for example to facilitate the infilling, and preferably concentrated in optional step i) before diafiltration. Preferably, the hydrogel is concentrated in optional step i) starting from a concentration of the hydrogel in the range of from 3 to 30 mg/g, preferably from 5 to 20 mg/g, more preferably from 5 to 10 mg/g.

Preferably, the hydrogel is concentrated in optional step i) until the desired concentration is reached and at most to a maximum concentration of 70 mg/g. In particular the concentration of the hydrogel after the optional concentrating step i) is in the range of from 10 to 70 mg/g, preferably from 20 to 50 mg/g, more preferably from 20 to 35 mg/g.

Preferably, the diafiltration in step ii) is conducted at a concentration of the hydrogel in the range of from 10 to 70 mg/g, preferably from 20 to 50 mg/g, more preferably 20 to 35 mg/g, also preferably 23 to 70 mg/g. Typically, the concentration of the hydrogel (in particular the hydrogel made of hyaluronic acid) is maintained at nearly the same level during diafiltration step ii).

Preferably, the diafiltration in step ii) is conducted until the amount of unwanted molecules (such as BDDE) in the hydrogel is below the limit of quantification. For example the diafiltration in step ii) is conducted for a period of from 1 to 8 hours, preferably 2 to 6 hours.

Preferably, the diafiltration in step ii) is conducted using a buffer solution, preferably a phosphate buffer solution, e.g. a buffer solution based on sodium phosphate, such as Na₂HPO₄ and NaH₂PO₄. In particular the diafiltration in step ii) is conducted using a buffer solution having a pH value in the range of 6 to 8, preferably 6.5 to 7.5.

Preferably the hydrogel is concentrated in step i) by applying a rotational speed in the range of from 70 1/min to 500 1/min, preferably from 70 1/min to 300 1/min, more preferably from 100 1/min to 300 1/min. Preferably the hydrogel is concentrated in step i) by a overpressure in the range of from 1 to 3 bar.

Preferably the diafiltration in step ii) is performed by applying a rotational speed in the range of from 70 1/min to 500 1/min, preferably from 70 1/min to 300 1/min, more preferably from 100 1/min to 300 1/min. Preferably the diafiltration in step ii) is performed by applying a overpressure in the range of from 1 to 3 bar.

In particular embodiments, the dynamic filtration device which is used in the method according to the invention is equipped with semipermeable filter disc(s) exhibiting a pore size of 5 nm to 2 μm, more particularly a pore size of 30 nm to 600 nm, more particularly a pore size of 80 nm to 300 nm, particularly a pore size of 5 nm to 60 nm.

In particular embodiments, the dynamic filtration device which is used in the method according to the invention is equipped with one or more semipermeable filter disc(s). In a preferred embodiment the dynamic filtration device is equipped with 1 to 10, preferably 4 to 6, semipermeable filter disc(s). The semipermeable filter discs may exhibit the same or different dimensions. It was surprisingly found that the number of semipermeable filter disc(s) can be increased up to 10 or preferably up to 6, without having negative impact on the viscosity and cross-linked structure of the hydrogel. Typically, shear forces increase with increasing number of discs and increasing rotational speed.

In particular embodiments, the dynamic filtration device is equipped with semipermeable filter disc(s) which are made of ceramic, metal or polymer material.

In particular embodiments, the biopolymer-based hydrogel is made of hyaluronic acid and the gel is concentrated by applying a rotational speed within the range of about 20 1/min to 150 1/min and a pressure within the range of about of about 0.5 to 2 bar to a final concentration of 10 to 30 mg/g and wherein the diafiltration is performed by applying a rotational speed within the range of 20 1/min to 150 1/min and a pressure within the range of 0.5 to 2 bar.

In a preferred embodiment, the biopolymer-based hydrogel is made of hyaluronic acid, the diafiltration in step ii) is performed by applying a rotational speed within the range of from 20 1/min to 500 1/min, preferably from 70 to 300 1/min and a pressure within the range of from 0.5 to 3 bar, preferably from 1 to 3 bar, and the diafiltration in step ii) is conducted at a concentration of the hydrogel in the range of from 10 to 70 mg/g, preferably from 20 to 50 mg/g, more preferably from 20 to 35 mg/g.

In a preferred embodiment, the biopolymer-based hydrogel is made of hyaluronic acid, the gel is pumped directly into the process chamber of the dynamic filtration device in step (i) (i.e. no optional concentrating step) and the diafiltration in step ii) is performed by applying a rotational speed within the range of 70 1/min to 500 1/min, preferably from 70 to 300 1/min, and a pressure within the range of 1 to 3 bar, wherein the diafiltration in step ii) is conducted at a concentration of the hydrogel in the range of from 10 to 70 mg/g, preferably 20 to 35 mg/g, and wherein the dynamic filtration device is equipped with 1 to 10, preferably 4 to 6, semipermeable filter disc(s).

During or after diafiltration of the hydrogel further substances may be added to the gel, for example: salts, buffer substances, vitamins (e.g. vitamin E, C, B6), antioxidant (e.g. ascorbic acid or derivatives thereof, zinc oxide), polyols (e.g. glycerol, mannitol), tri-calcium phosphate particles (e.g., alpha- and beta-tri-calcium phosphate and hydroxyapatite particles), agents (e.g. anesthetics, anti-inflammatory agents, stimulus-reducing agents, vasoconstrictive agents or vasodilatory agents, anticoagulants, humidity-providing substances, immuno-suppressives, antibiotics, etc.) as well as growth factors, peptides or proteins (e.g. neurotoxins). In particular, an anesthetic such as lidocaine may be added to the gel. Preferably, lidocaine, is contained in the gel in a concentration of from 0.05 to 5.0 wt.-%, 0.1 to 2.0 wt. %, 0.1 to 1.0 wt.-%, 0.1 to 0.5 wt.-%, 0.1 to 0.4 wt.-%, 0.2 to 0.4 wt.-% or from 0.2 to 0.3 wt.-%, based on the total weight of the composition.

Preferably the optional concentrating in step i) and/or the diafiltration in step ii) is performed at a temperature in the range of 15° C. to 80° C., preferably 20° C. to 70° C.

In a preferred embodiment the diafiltration in step ii) and optionally the concentration step i) are performed at a temperature in the range of from 60° C. to 70° C., preferably at a temperature in the range of from 60° C. to 70° C. and for a time period of from 2 to 4 hours (e.g. 3.5 hours). In particular a temperature in the range of 60° C. to 70° C. is advantageous due to the decreased viscosity of the hydrogel.

In particular embodiments, in step a) the gel is additionally undergoing a thermal treatment to reduce unwanted molecules, wherein 60° C. are applied for up to four hours.

The dynamic filtration device may comprise a sterilization means, preferably pure steam, for rinsing the device and/or the passage with steam, a sterilization fluid, in particular washing liquid, preferably filtered water, and/or sterilized/particle-free air. The sterilization means may comprise a supply unit comprising sterilization fluid, and a storage unit for sterilization fluid.

In particular embodiments the gel is sterilized in the dynamic filtration device at 121° C. to 135° C. with a holding time of about 2 to 30 minutes.

In particular embodiments, the method according to the invention is conducted in less than 10 hours, in particular in less than 5 hours.

The method according to the invention may additionally comprise the following steps:
i) degassing the gel to remove air bubbles under vacuum (20 to 200 mbar),
ii) filling the gel into syringes,
iii) sterilize the gel in the syringe by heat in an autoclave.

In another aspect the present invention relates to a cross-linked biopolymer-based hydrogel obtainable by the method according to the invention.

In another aspect the present invention relates to a use of the cross-linked biopolymer-based hydrogel obtained by the method according to the invention for aesthetic applications, in particular soft tissue augmentation.

### EXAMPLES

In the following, the method according to the invention is explained in more detail.

### Example 1: Method Including Step of Diluting, Concentrating and Diafiltration of the Gel

A hyaluronic acid hydrogel which was cross-linked with BDDE (cf. for example WO 2005/085329) and exhibits an initial concentration of 24 mg/g was diluted in a range between 1:4 to 1:8 using a buffer containing Na2HPO4*2H2O: 0.994 g/L; NaH2PO4*2H2O: 0.51 g/L; Mannitol: 42 g/L and water for injection. The diluted gel was transferred to a container, connected to a dynamic filtration device inlet via a pipe or tubing. The dynamic filtration device consisted of a Krauss Maffei Dynamic Crossflow Filter DCF 152/S (Andritz AG) having one filter disc (0.034 m² filter area, 152 mm diameter and a pore size of 7 nm), one container as gel reservoir and a second container as buffer reservoir. Both containers were connected to the inlet port of the DCF 152/S via tubing and ball valve to select the respective medium in the different process steps. The double jacket of the housing of the DCF 152/S was connected to a refrigerated circulator to maintain the temperature in the double jacket at a preset temperature of 20° C. The filtrate of the DCF 152/S was collected in a container, placed on a balance to determine the amount of filtrate. The following steps were applied:
i) concentrating the gel by applying pressure (1.5 bar) to the closed gel container, containing the diluted gel, open the way to the inlet port of the DCF 152/S and removing the filtrate via the filter discs and hollow shaft (see WO 00/47312). The filter disc was rotating with 150 rpm. The gel was concentrated until the concentration of hyaluronic acid in the gel was 24 mg/g.
ii) conducting a diafiltration with a buffer solution containing Na₂HPO₄*2H₂O: 0,994 g/L; NaH₂PO₄*2H₂O: 0.51 g/L; Mannitol: 42 g/L and water for injection by applying pressure (1.5 bar) to the closed buffer container, containing the buffer, open the way to the inlet port of the DCF 152/S and removing the filtrate via the filter discs and hollow shaft (see WO 00/47312). The buffer solution may contain lidocaine (0.27-0.33 w/w %). As an alternative, lidocaine may be added after diafiltration. The filter disc was rotating with 150 rpm. The gel was filtered until BDDE is present below the limit of quantification.

The amount of BDDE was determined according to the following method:
1. Sample preparation:
   a. Enzymatic degradation of HA HA was degraded by addition of hyaluronidase solution and subsequent incubation for 1 to 4 hours under slight shaking until viscosity was reduced.
   b. Extraction of BDDE
      BDDE was extracted from the degraded sample (described under a) by addition of ethylacetate (5% v/v) and orbital shaking for 20 min at 400 rpm. The solution was centrifuged at 10 min at 5000 rpm to separate the organic and aqueous phase. The organic phase, containing the BDDE was transferred to a GC-vial.
2. Gaschromatographic determination
   a. 5 μL of the sample (described under 1 b) was injected in a GC (split injector, split ration 1:5, 250° C.) and separated on a DB-1 column (Agilent DB-1, 30 m, 0.25 mm ID, 0.25 μm film) using a temperature ramp 100° C.-40° C./min-280° C. and final holding at 280° C. for 30 sec. A FID was used as detector.
   b. Data evaluation was performed by an internal standard method according to European Pharmacopoeia Chapter 2.2.46.

The result of one experiment according to Example 1 is shown in the following table 1:

TABLE 1

| | | | | BDDE Gel | BDDE DCF |
|---|---|---|---|---|---|
| | | | | BDDE content | Final BDDE |
| Disc | Pressure | T | Duration | at beginning | content |
| [nm] | [bar] | [° C.] | [h:m] | [ppm] | [ppm] |
| 7 | 1.5 | 20 | 6:00 | >150 | below limit of |
| | | | | | quantification |
| | | | | | (LOQ) |

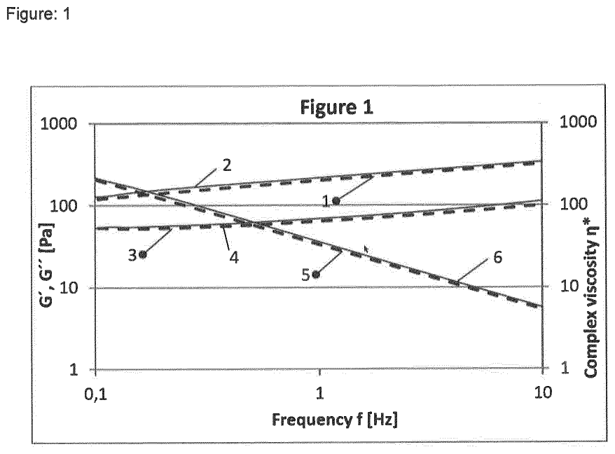

The structural stability of a gel was maintained during the method according to the invention as can be seen from FIG. 1 which shows a comparison of two different gels. One gel was treated according to the invention (“DCF gel”) and the other gel was treated with a standard method (“standard gel”).

The continuous lines represent the characteristic progression of G′, G″ and q of a gel after dialysis which was produced according to a standard method, whereas the dotted lines represent the characteristic progression of a gel which was processed according to the invention.

It is apparent that there were no significant differences regarding the curve progressions shown in the frequency test which is commonly used for the characterization of gels. The curves related to the gel according to the invention are slightly below the curves related to the standard gel because the HA-concentration of the gel is slightly lower. With regard to the structural stability of the gel, the parallel increase of G′ and G″ (G′>G″) clearly indicates that the gel has maintained its cross-linked structure during the method according to the invention.

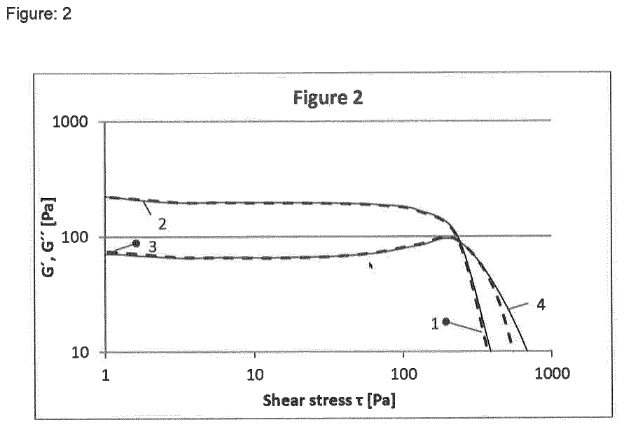

In addition, it is shown in FIG. 2 that the flow-point of both gels (“DCF gel” and standard gel”; see above) is identical which further indicates that structural stability of the gel processed according to the invention was maintained.

### Example 2: Method of Diafiltrating the Gel without Step of Diluting the Gel

A hyaluronic acid hydrogel which was cross-linked with BDDE (cf. for example WO 2005/085329) and exhibits a concentration of 32-35 mg/g was pumped directly into the process chamber of the dynamic filtration device. The gel contained 226.8 ppm unbound BDDE. The dynamic filtration device consisted of a Krauss Maffei Dynamic Crossflow Filter DCF 152/S (Andritz AG) having one filter disc (0.034 m² filter area, 152 mm diameter and a pore size of 5 nm), one container as gel reservoir and a second container as buffer reservoir. Both containers were connected to the inlet port of the DCF 152/S via tubing and ball valve to select the respective medium in the different process steps. The double jacket of the housing of the DCF 152/S was connected to a refrigerated circulator to maintain the temperature in the double jacket at a preset temperature of 20° C. The filtrate of the DCF 152/S was collected in a container, placed on a balance to determine the amount of filtrate. The following step was applied:
i) conducting a diafiltration with a buffer solution containing Na₂HPO₄*2H₂O: 5.96 g/L; NaH₂PO₄*2H₂O: 2.57 g/L; NaCl: 2.29 g/L and water for injection by applying pressure (1.5 bar) to the closed buffer container, containing the buffer, open the way to the inlet port of the DCF 152/S and removing the filtrate via the filter discs and hollow shaft (see WO 00/47312). The buffer solution may contain lidocaine (0.27-0.33 w/w %). As an alternative, lidocaine may be added after diafiltration. The filter disc was rotating with 150 rpm (1/min). The gel was filtered until unbound BDDE was present below the limit of quantification (LOQ). The amount of BDDE was determined as explained above (see Example 1). The dynamic filtration process was performed within 2 hours and 1 minute.
   The results of further experiments are shown in the following table 2:

TABLE 2

| | | | | | BDDE Gel | BDDE DCF |
|---|---|---|---|---|---|---|
| | | | | | BDDE content | Final BDDE |
| | Disc | Pressure | T | Duration | at beginning | content |
| Trial | [nm] | [bar] | [° C.] | [h:m] | [ppm] | [ppm] |
| 1 | 5 | 1.5 | 20 | 03:17 | 226.8 | below limit |
| | | | | | | of quanti- |
| | | | | | | fication |
| | | | | | | (LOQ) |
| 2 | 30 | 1.5 | 20 | 02:18 | 226.8 | below LOQ |
| 3 | 2000 | 1.5 | 20 | 02:01 | 226.8 | below LOQ |

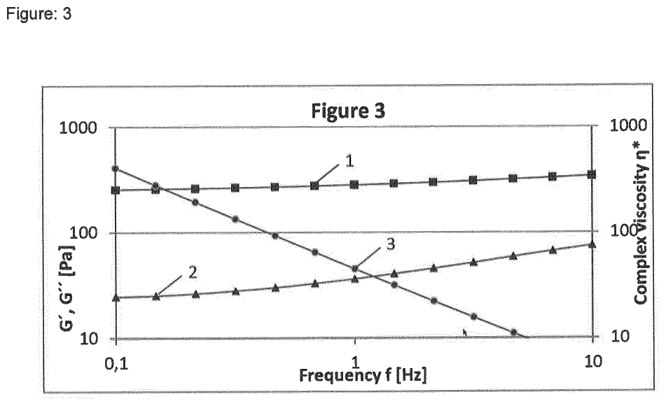

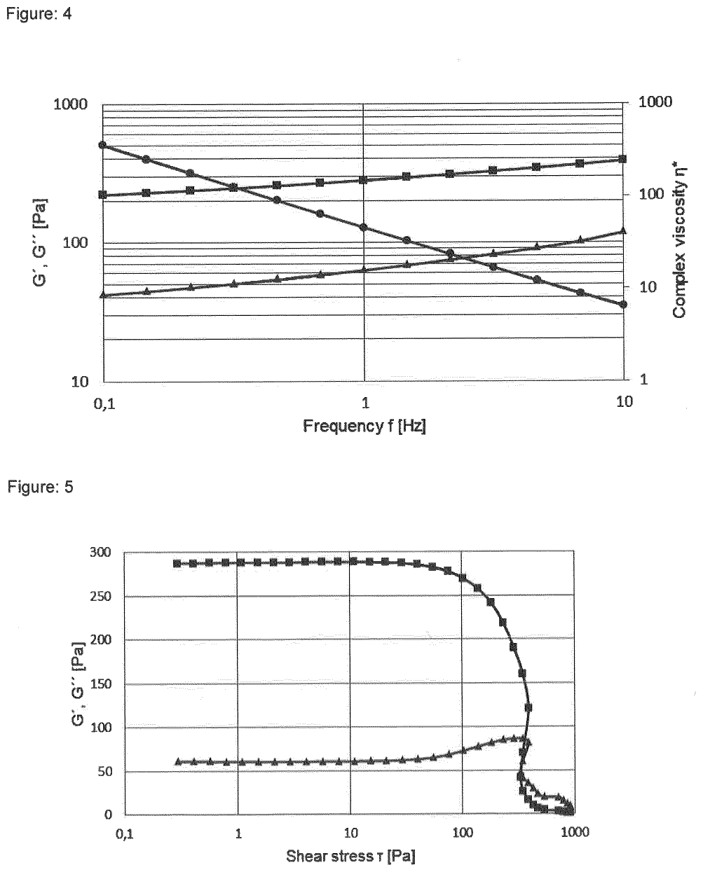

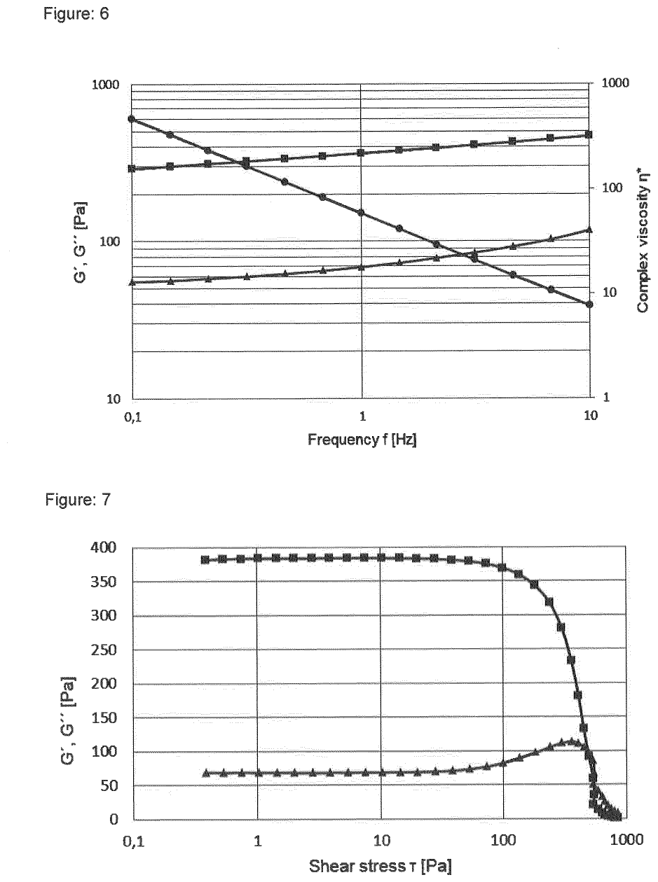

A further example of the fact that the structural stability of the gel was maintained during the method according to the invention can be seen in FIG. 3. After the method according to Example 2 was applied, the gel was analyzed with the help of a frequency test which is commonly used for the characterization of gels. The used frequency range was 0.1 to 10 Hz. The parallel increase of G′ and G″ (G′>G″) clearly indicates that the gel (“DCF gel”) has maintained its cross-linked structure during the method according to Example 2.

### Example 3: Method of Diafiltratinq the Gel without Step of Diluting the Gel and Using Dynamic Filtration Device with Six Filter Discs

The example 3 was carried out following the example 2 described above with the difference that the dynamic filtration device used was a Krauss Maffei Dynamic Crossflow Filter DCF 152/S (Andritz AG) having six (instead of one as in examples 1 and 2) filter disc (0.034 m² filter area, 152 mm diameter and a pore size of 5 nm). A hyaluronic acid hydrogel, which was cross-linked with BDDE (cf. for example WO 2005/085329) and exhibits a concentration of 66 mg/g, was diluted with the factor 1:2 using a buffer solution as described in examples 1 and 2. The hyaluronic acid hydrogel having a concentration of about 33 mg/g was pumped directly into the process chamber of the dynamic filtration device. The gel contained 226.8 ppm unbound BDDE.

The diafiltration was carried out as described in example 2, wherein the differing process conditions are given in table 3. The diafiltration was carried out until BDDE is present below the limit of quantification and typically until the volume of the filtrate was seven times of the volume of the chamber of the filtration device.

TABLE 3

| | Disc | | | Rotational | | | BDDE | BDDE |
|---|---|---|---|---|---|---|---|---|
| | pore size | Number | Pressure | speed | T | Duration | Gel | DCF |
| Trial | [nm] | of discs | [bar] | [1/min] | [° C.] | [h:m] | [ppm] | [ppm] |
| 4 | 5 | 6 | 2 | 177 | 45 | 04:23 | 226.8 | below LOQ |
| 5 | 5 | 6 | 3 | 77 | 70 | 07:50 | 226.8 | below LOQ |

LOQ: limit of quantification
BDDE Gel: BDDE content at beginning
BDDE DCF: Final BDDE content in the hydrogel after filtration

The structural stability of the gel was maintained during the DCF method according to trials 4 and 5 according to example 3 as shown in the FIGS. 4 (trial 4) and 5 (trial 5). After the method according to Example 3 was applied, the gels were analyzed with the help of a frequency test which is commonly used for the characterization of gels. The used frequency range was 0.1 to 10 Hz. The parallel increase of G′ and G″ (G′>G″) clearly indicates that the gels have maintained their cross-linked structure during the method according to Example 3 (trials 4 and 5).

It was surprisingly found that the structure of the hydrogel is not effected negatively (i.e. the cross-linking structure is maintained), when the number of filter discs as well as the rotational speed were increased compared to example 2. Typically, shear forces increase with increasing number of discs and increasing rotational speed.

1-15. (canceled)

16. A method for dynamic filtration of a cross-linked biopolymer-based hydrogel comprising the following steps:
a) transferring a cross-linked biopolymer-based hydrogel in a dynamic filtration device which is equipped with at least one semipermeable filter disc and diafiltrating the gel comprising the steps of:
i) concentrating the gel by applying a rotational speed within the range of 20 1/min to 500 1/min and an overpressure within the range of 0.5 to 6 bar to a predetermined concentration; or pumping the gel directly into the process chamber of the dynamic filtration device; and
ii) conducting a diafiltration to reduce unwanted molecules by applying a rotational speed within the range of 20 1/min to 500 1/min and an overpressure within the range of 0.5 to 6 bar; and
b) optionally adding a mixture comprising a non-cross-linked polymer and water to the gel.

17. The method of claim 16, wherein the biopolymer-based hydrogel is made of a polymer which is selected from the group consisting of hyaluronic acid, heparosan, alginate, pectin, gellan gum, chondroitin sulfate, keratan, keratan sulfate, heparin, heparin sulfate, cellulose, chitosan, carrageenan, xanthan, and salts or derivatives thereof, and combinations thereof.

18. The method of claim 16, wherein the hydrogel is cross-linked by a chemical cross-linking agent, and the hydrogel contains a surplus of the chemical cross-linking agent, wherein the surplus of the chemical cross-linking agent and/or other unwanted molecules are removed.

19. The method of claim 16, wherein the at least one semipermeable filter disc exhibits a pore size of 5 nm to 2 μm.

20. The method of claim 16, wherein the at least one semipermeable filter disc exhibits a pore size of 30 nm to 600 nm.

21. The method of claim 16, wherein the at least one semipermeable filter disc exhibits a pore size of 80 nm to 300 nm.

22. The method of claim 16, wherein the at least one semipermeable filter disc exhibits a pore size of 5 nm to 60 nm.

23. The method of claim 16, wherein the dynamic filtration device is equipped with 1 to 10 semipermeable filter disc(s).

24. The method of claim 16, wherein the at least one semipermeable filter disc is made of ceramic, metal, or polymer material.

25. The method of claim 16, wherein the hydrogel is cross-linked by 1,4-butanediol diglycidyl ether (BDDE), and the hydrogel contains a surplus of BDDE, wherein the surplus of BDDE and/or other unwanted molecules are removed.

26. The method of claim 16, wherein the biopolymer-based hydrogel is made of hyaluronic acid, the diafiltration in step ii) is performed by applying a rotational speed within the range of 20 1/min to 500 1/min and a pressure within the range of 0.5 to 3 bar, and the diafiltration in step ii) is conducted at a concentration of the hydrogel in the range of 10 to 70 mg/g.

27. The method of claim 16, wherein during or after diafiltration an anesthetic agent is added to the gel.

28. The method of claim 27, wherein during or after diafiltration lidocaine is added to the gel.

29. The method of claim 16, wherein the diafiltration in step ii) and optionally the concentration step i) are performed at a temperature in the range of from 60° C. to 70° C. and for a time period of from 2 to 4 hours.

30. The method of claim 16, wherein the gel is sterilized in the device at 121° C. to 135° C. with a holding time of about 2 to 30 minutes.

31. The method of claim 16, wherein the method is conducted in less than 10 hours.

32. The method of claim 16, wherein the method is conducted in less than 5 hours.

33. A cross-linked biopolymer-based hydrogel obtainable by the method of claim 16.

34. A method for utilizing the hydrogel of claim 33 comprising aesthetic application of the hydrogel to soft tissue.

35. The method of claim 34, wherein the aesthetic application is soft tissue augmentation.
